(19) United States
(12) Patent Application Publication
(10) Pub. No.: US 2006/0110373 A1
(43) Pub. Date: May 25, 2006

(54) PTU compounds for promoting the in vitro culture of (highly pigmented) melanocytes

(75) Inventors: Marcelle Regnier, Paris (FR); Claire Tremblaye, Asnieres (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: L'OREAL, Paris (FR)

(21) Appl. No.: 11/270,642

(22) Filed: Nov. 10, 2005

Related U.S. Application Data
(60) Provisional application No. 60/643,331, filed on Jan. 13, 2005.

(30) Foreign Application Priority Data
Nov. 10, 2004 (FR) 04/12001

(51) Int. Cl. A61K 35/36; C12N 5/08

(52) U.S. Cl. 424/93.7; 435/371

(57) ABSTRACT

A method for promoting the in vitro multiplication of human melanocytes and/or the freezing thereof, notably human melanocytes obtained from individuals of phototype IV, V or VI or from hyperpigmentary lesions, entails introducing into a culture of same, a thus effective amount of at least one 1-phenyl-3-(2-thiazolyl)-2-thiourea (PTU) compound or PTU analogue or PTU mimetic selected from the group consisting of vitamin C, arbutin, hydroquinone, kojic acid or acid or ester derivative thereof, ellagic acid, aminophenol derivative, procysteine or derivative thereof, niacinamide, isothiocyanate, thiocyanate, lucinol and mixtures thereof; also provided thereby can be melanocyte libraries, co-cultures and reconstructed epidermides and/or reconstructed skin that are highly pigmented which are useful for the study of pigmentation disorders, for the screening of cosmetic or dermatological active agents, or for the treatment of skin lesions, in particular in individuals with a high phototype.

### CROSS-REFERENCE TO PRIORITY/PROVISIONAL APPLICATIONS

This application claims priority under 35 U.S.C. § 119 of FR 04/12001, filed Nov. 10, 2004, and of provisional application Ser. No. 60/643,331, filed Jan. 13, 2005, each hereby expressly incorporated by reference and each assigned to the assignee hereof.

### BACKGROUND OF THE INVENTION

1. Technical Field of the Invention

The present invention relates to the cellular and tissue engineering of melanocytes, in particular of melanocytes derived from individuals of phototypes IV, V or VI or from hyperpigmentary lesions.

The present invention also relates, in particular, to the use of at least one compound selected from among 1-phenyl-3-(2-thiazolyl)-2-thiourea (PTU), a PTU analogue, a PTU mimetic and mixtures thereof, for promoting the in vitro multiplication of human melanocytes and/or the freezing thereof, and by the same token, the use of said melanocytes for obtaining co-cultures (for example: melanocyte-keratinocyte co-cultures) or reconstructed epidermides and/or reconstructed skin.

In particular, this invention relates to the use of PTU for promoting the in vitro multiplication of human melanocytes derived from individuals of phototype IV, V or VI or from hyperpigmentary lesions, and also to methods for obtaining libraries of melanocytes, co-cultures and reconstructed epidermides and/or reconstructed skin, suited, in particular, for the study of pigmentation disorders, for the screening of active agents (for example: depigmenting agents) or for the treatment of skin lesions, in particular in individuals who are of high phototype, referred to as “highly pigmented”. The present invention also relates to the corresponding production kits.

2. Description of Background and/or Related and/or Prior Art

Skin phototypes are defined by means of the Fitzpatrick classification, which is based on the skin's reactivity to the effects of solar radiation:
I always burns, never tans
II always burns, minimal tanning
III burns mildly, tans gradually
IV burns minimally, tans very easily
V rarely burns, tans profusely
VI never burns, highly pigmented

The present invention is particularly focused on melanocytes derived from individuals who are of phototypes IV, V or VI or derived from hyperpigmentary lesions, described as “highly pigmented melanocytes”. Hereinafter, reference will also be made to “highly pigmented epidermides and/or highly pigmented skin”.

These phototypes IV, V and VI correspond in particular to the phototypes of populations of African, North African, Indian, Asian (for example: Chinese, Korean, Japanese, Indonesian) and Afro-American ethnic origin. Certain individuals of Caucasian origin also have a high phototype (IV).

Hyperpigmentary lesions, which are characterized by the presence of highly pigmented melanocytes, can on the other hand affect individuals of any phototype.

The color of human skin depends on various factors, and in particular on the seasons of the year, on the ethnic origin and on the sex; it is mainly determined by the nature and the concentration of melanin produced by the melanocytes.

Melanocytes are specialized cells which, by means of specific organelles, melanosomes, synthesize melanin according to the following scheme:
(Tyrosinase) (Tyrosinase) Tyrosine→Dopa→Dopaquinone→Dopachrome→Melanin

The differences in skin color between individuals are due to the number, to the size, to the type, to the distribution and to the degradation of melanosomes, and also to the activity of tyrosinase. The number of melanosomes is approximately the same among all individuals. However, the light phototypes I to III (Caucasians) synthesize eumelanosomes and pheomelanosomes, whereas Negroid individuals synthesize mainly eumelanosomes. In addition, tyrosinase activity is greater in Negroid skin than in Caucasian skin.

Environmental factors also play an important role in the coloration of the skin. Thus, it is known that solar radiation is capable of inducing neomelanogenesis that results in browning of the skin (tanning), but also pigmentary disorders (for example: hyperpigmented spots). This browning may be aesthetically embarrassing, in particular for certain Asian populations.

Skin referred to as “highly pigmented” or skin with a high phototype according to the invention corresponds in particular to populations of African, North African, Indian, Asian (for example: Chinese, Korean, Japanese, Indonesian) and Afro-American origin, which arouse an increasing scientific interest for the acquisition of new knowledge and the development of cosmetic and dermatological products suitable for these high phototypes.

Moreover, hyperpigmentary lesions, which can affect individuals of any phototype, also constitute an important field of research for understanding the mechanisms linked to these hyperpigmentary disorders and the development of suitable treatments.

There is therefore a need to develop novel cell and tissue models containing melanocytes, in particular “highly pigmented” melanocytes derived from an individual of phototype IV, V or VI or from hyperpigmentary lesions, that can be used as study models for the search for novel cosmetic and dermatological active agents, for the study of hyperpigmentary disorders, or are useful in particular to treat skin lesions (for example: burns, excision, naevus, tattoo, pigmentation disorders associated with a dermatological condition, etc.), in particular in individuals who have high phototypes.

However, it too is known that there are difficulties in culturing melanocytes in vitro, this being a minority cell type ( 1/35th of the number of keratinocytes, and slow replication) of low viability, in particular for highly pigmented melanocytes.

### SUMMARY OF THE INVENTION

It has now unexpectedly been determined that the use of 1-phenyl-3-(2-thiazolyl)-2-thiourea PTU (for example: 10 μM) on melanocytes of phototype VI (African) in culture makes it possible to:

promote the in vitro multiplication of African melanocytes; PTU is even capable of stimulating the in vitro multiplication of African melanocytes by a factor of 2 to 10 compared with melanocytes not treated with PTU; and

promote the freezing, thawing and re-culturing of African melanocytes by maintaining and/or increasing their percentage viability (for example, by a factor of 10 to 20 compared with melanocytes not treated with PTU) and maintaining their functionality, i.e., their ability to proliferate, to produce melanin and to integrate into a reconstructed epidermis.

It is known practice in the prior art to administer phenylthioureas, thioureas and monoxide or dioxide derivatives as depigmenting agents in cosmetic or pharmaceutical compositions, as described in particular in WO 2004/032912 and WO 01/64206.

However, to the knowledge of the present inventors, the use of a depigmenting agent, and in particular that of 1-phenyl-3-(2-thiazolyl)-2-thiourea (PTU), for promoting the in vitro multiplication of melanocytes and/or the freezing thereof, in particular of highly pigmented melanocytes, has never been described or suggested.

The present invention therefore features the use of at least one compound selected from among 1-phenyl-3-(2-thiazolyl)-2-thiourea (PTU), a PTU analogue, a PTU mimetic and mixtures thereof, for promoting the in vitro multiplication of human melanocytes and/or the freezing thereof.

In particular, the compound selected from among PTU, a PTU analogue, a PTU mimetic and mixtures thereof is suited to promote the in vitro multiplication of human melanocytes derived from individuals of phototype IV, V or VI or from hyperpigmentary lesions.

The human melanocytes employed according to the invention may, for a given phototype, be derived from samples taken from one or more individuals. Preferably, said individuals will be of the same ethnic origin, but they may also be of different ethnic origins and have the same phototype.

The samples will generally be taken from the same anatomical site, but they may also be taken from different anatomical sites. In addition, according to an alternative, individuals of the same age or individuals of different ages may be selected.

For the case of individuals suffering from hyperpigmentary lesions, the melanocytes may be taken from the normal areas of the individual, for the purpose of treating said lesions, or directly from the lesioned areas, for the purpose of furthering the knowledge regarding hyperpigmentary disorders.

### DETAILED DESCRIPTION OF BEST MODE AND SPECIFIC/PREFERRED EMBODIMENTS OF THE INVENTION

The term “PTU analogue” according to the invention means, in particular, a compound selected from among thioureas, their phenyl, monoxide or dioxide derivatives, and the compounds of general formula (I): 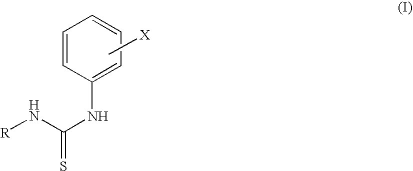
wherein:

X is a hydrogen atom or a linear or branched C₁-C₄ alkyl radical or a halogen or a cyano group or a radical —OR′ or COOR′ in which R′ is a hydrogen atom or a C₁-C₄ alkyl radical, and

R is a hydrogen atom or an aromatic group that is unsubstituted or substituted with groups equivalent to X.

In particular, a compound of formula (I) is employed wherein X is a hydrogen atom and R is a 2-thiazolyl group, i.e., 1-phenyl-3-(2-thiazolyl)-2-thiourea (PTU).

As examples of monoxide or dioxide derivatives of thiourea, representative are those described in WO 2004/032912, and as examples of phenylthiourea derivatives, representative are those described in WO 01/64206.

The term “PTU mimetic” according to the invention means, in particular, a compound selected from among vitamin C, arbutin, hydroquinone, kojic acid or its acid or ester derivatives, ellagic acid, aminophenol derivatives, procysteine and derivatives, niacinamide, isothiocyanate and thiocyanate, lucinol, any other tyrosinase inhibitor or any agent capable of inhibiting melanin production, whatever the mechanism involved (for example: action on the enzymes for melanin production and/or on melanosome transfer).

The PTU compound, PTU analogue or PTU mimetic will be used in culture in vitro according to the invention in an effective and non-cytotoxic amount, i.e., in an amount capable of promoting the in vitro multiplication of the melanocytes without affecting their viability.

In particular, this effective and non-cytotoxic amount of PTU, PTU analogue or PTU mimetic may be determined according to the following method:

a) a compound (for example: PTU) is applied to melanocytes in culture, in various amounts; the melanocytes are preferably melanocytes of phototype IV, V or VI;

b) the cytotoxicity of the various amounts of PTU on the melanocytes in culture is evaluated by determining the percentage of cells that adhere to the culture support compared with the control (in the absence of PTU), and the amounts of PTU for which the percentage of cells that adhere to the culture support is barely or not at all decreased compared with the control, or even increased compared with a control, are selected; in particular, the percentage of cells that adhere to the culture support will be greater than or equal to 60%, preferentially greater than or equal to 70%, and even more preferentially greater than or equal to 80%;

c) the effectiveness of the non-cytotoxic amounts of PTU selected in b), on the proliferation of melanocytes in culture, is evaluated by quantifying the number of cells obtained after each passage compared with the control (in the absence of PTU), and the amounts of PTU for which an increase in the multiplication of said melanocytes is obtained at each passage, compared with the control, are selected; in particular, the amounts of PTU capable of stimulating the multiplication of melanocytes in culture by a factor of 2 to 10, compared with the control, are selected.

Preferably, such an effective and non-cytotoxic amount of a compound selected from among 1-phenyl-3-(2-thiazolyl)-2-thiourea (PTU), a PTU analogue, a PTU mimetic and mixtures thereof is less than the concentration of said compound that is required to inhibit melanin synthesis by 50% (IC50), preferably the effective amount will be from IC50/1.5 to IC50/10.

In particular, the effective and non-cytotoxic amount of PTU according to the invention may range from 2 μM to 40 μM, preferably from 2 μM to 20 μM. A concentration of PTU equal to 2 μM, 5 μM, 10 μM, 15 μM or 20 μM may, for example, be used.

The compound selected from among 1-phenyl-3-(2-thiazolyl)-2-thiourea (PTU), a PTU analogue, a PTU mimetic and mixtures thereof is present in the in vitro culture according to the invention in an effective and non-cytotoxic amount capable of stimulating the in vitro multiplication of human melanocytes, in particular of phototype IV, V or VI, by a factor of 2 to 10 compared with non-treated human melanocytes.

Indeed, it has in fact now been shown that the addition of a concentration of 5 μM or 10 μM of PTU at passage 2/3 of African human melanocytes in culture is capable of stimulating, by a factor of 5 at passage 8 and by a factor of 15 at passage 9, the multiplication of the African human melanocytes compared with non-treated African human melanocytes, which themselves multiply 2-fold, in a constant manner, at each passage. These passages correspond to a step consisting of trypsinization and re-culturing of the melanocytes that have reached confluency.

This amount of PTU may be added during a culture passage (for example: step consisting of trypsinization and re-culturing), but also at any time during the culture of the melanocytes.

The in vitro use of at least one compound selected from among 1-phenyl-3-(2-thiazolyl)-2-thiourea (PTU), a PTU analogue, a PTU mimetic and mixtures thereof is also useful for maintaining and/or increasing the percentage viability of human melanocytes, in particular derived from individuals of phototype IV, V or VI or from hyperpigmentary lesions, after freezing, thawing and re-culturing.

In particular, the compound selected from among 1-phenyl-3-(2-thiazolyl)-2-thiourea (PTU), a PTU analogue, a PTU mimetic and mixtures thereof, brought into contact, in an effective amount, with human melanocytes before freezing, is capable of increasing the percentage viability of the human melanocytes, in particular the highly pigmented human melanocytes, after thawing and re-culturing, by a factor of 10 to 100, in particular by a factor of 10 to 20, compared with the percentage viability of non-treated human melanocytes.

In particular, the viability of the highly pigmented melanocytes (for example: African) treated with an effective and non-cytotoxic amount of PTU, for example a concentration of 5 μM or 10 μM, will be at least 50%, and preferentially at least 70%. In particular, it is 80% with 10 μM PTU, compared with the viability of non-treated African melanocytes, which is only 5% after thawing and re-culturing.

The in vitro use of at least one compound selected from among 1-phenyl-3-(2-thiazolyl)-2-thiourea (PTU), a PTU analogue, a PTU mimetic and mixtures thereof, in an effective and non-cytotoxic amount, capable of (i) stimulating the in vitro multiplication of human melanocytes, in particular of highly pigmented melanocytes, and (ii) maintaining and/or increasing their percentage viability after freezing, thawing and re-culturing, may advantageously be used in methods for obtaining cell cultures or cell libraries, co-cultures (for example: melanocyte-keratinocyte co-cultures) and/or methods for obtaining organotypic skin models, in particular highly pigmented models.

The invention therefore also features the use of at least one compound selected from among 1-phenyl-3-(2-thiazolyl)-2-thiourea (PTU), a PTU analogue, a PTU mimetic and mixtures thereof, in a method for obtaining human melanocyte cultures or human melanocyte libraries, in particular cultures of human melanocytes or libraries of human melanocytes derived from individuals of phototype IV, V or VI or from hyperpigmentary lesions.

These melanocyte libraries may be primary melanocyte libraries or secondary melanocyte libraries.

The term “melanocyte culture” according to the invention means, in particular, a preparation of melanocytes derived from a natural tissue, cultured in vitro and kept alive under artificial conditions (ex vivo, in vitro), in the presence of PTU.

The term “melanocyte library” according to the invention means, in particular, a preparation of melanocytes that has been conserved for the purpose of subsequent use. The cells may, for example, be frozen and conserved in the form of aliquots. The cells may originate from natural tissues of any phototype and may be either directly stored in the presence of PTU, for example in frozen form, or multiplied in vitro in the presence of PTU before being stored, or may be stored in the absence of PTU and placed in the presence of PTU after thawing.

Advantageously, the melanocytes derived from individuals of high phototype IV, V or VI or from hyperpigmentary lesions will be placed in the presence of PTU before and/or during freezing.

Melanocytes of light phototype may be directly stored in the absence of PTU and multiplied in the presence of PTU after thawing.

The term “primary melanocyte library” means a melanocyte library obtained after a cycle comprising a multiplication step and a freezing step.

The term “secondary melanocyte library” means a melanocyte library obtained after at least two successive multiplication-freezing cycles.

The preparation of a culture or of a library of human melanocytes, in particular highly pigmented human melanocytes, may, for example, entail:

a) a step of preparation of the human melanocytes derived from individuals of any phototype, in particular derived from individuals of phototype IV, V or VI or from hyperpigmentary lesions;

b) a step of multiplication of the melanocytes in a culture medium suitable for culturing melanocytes, in the presence or absence of at least one compound selected from among 1-phenyl-3-(2-thiazolyl)-2-thiourea (PTU), a PTU analogue, a PTU mimetic and mixtures thereof (obtaining a primary melanocyte culture);

c) optionally, a step of freezing of said melanocytes (obtaining a primary melanocyte library), in the presence or absence of at least one compound selected from among 1-phenyl-3-(2-thiazolyl)-2-thiourea (PTU), a PTU analogue, a PTU mimetic and mixtures thereof, for the purpose of subsequent use;

d) optionally, a secondary multiplication step, in the presence of at least one compound selected from among 1-phenyl-3-(2-thiazolyl)-2-thiourea (PTU), a PTU analogue, a PTU mimetic and mixtures thereof, followed by a freezing step (obtaining a secondary melanocyte library).

In the case of melanocytes derived from individuals of high phototype IV, V or VI or from hyperpigmentary lesions, the multiplication step b) will advantageously be carried out in the presence of at least one compound selected from among 1-phenyl-3-(2-thiazolyl)-2-thiourea (PTU), a PTU analogue, a PTU mimetic and mixtures thereof.

According to one particular embodiment, the PTU, a PTU analogue and/or a PTU mimetic may be added as soon as step a) of preparation of said human melanocytes.

Step a) of preparation of the human melanocytes may comprise the following steps:

taking a sample of skin tissue from individuals of the same phototype, selected from phototypes I to VI, in particular IV to VI; or taking a sample of skin tissue from hyperpigmentary lesions;

separating the epidermis from the dermis, for example by trypsinization;

centrifuging and taking up the cells (melanocytes and keratinocytes), for example in foetal calf serum;

seeding at a density of 12 to 15×10⁶ cells per 75 cm² flask, in medium suitable for culturing melanocytes (for example: M2, Promocell), in the presence or absence of 10 μM PTU, for example;

purifying the melanocytes by detachment from the support, for example in the presence of trypsin-EDTA, and taking up in an equivalent volume of culture medium for melanocytes (for example: M2, Promocell), in the presence or absence of 10 μM PTU, for example;

seeding said melanocytes at a density of 10⁶ cells per 75 cm² flask, in culture medium for melanocytes (for example: M2, Promocell), in the presence or absence of 10 μM PTU, for example.

A primary culture of human melanocytes, for example of phototype VI (African) is thus obtained, in which the melanocytes are 80% viable, i.e., 80% of the melanocytes adhere to the culture flask or support.

For a given phototype, the melanocytes may be derived from samples taken from one or more individuals. Preferably, said individuals will be of the same ethnic origin, but they may also be of different ethnic origins and have the same phototype. The samples will generally be taken from the same anatomical site, but they may also be taken from different anatomical sites. In addition, according to an alternative embodiment, individuals of the same age or individuals of different ages may be selected.

Step b) of multiplication of the melanocytes may be carried out in culture medium for melanocytes, in the presence or absence of PTU, of a PTU analogue or of a PTU mimetic.

In the case of melanocytes derived from individuals of high phototype IV, V or VI or from hyperpigmentary lesions, the multiplication step b) will advantageously be carried out in the presence of at least one compound selected from among 1-phenyl-3-(2-thiazolyl)-2-thiourea (PTU), a PTU analogue, a PTU mimetic and mixtures thereof.

PTU may, for example, be added at a concentration ranging from 2 to 40 μM, preferably ranging from 2 to 20 μM, and in particular a concentration of 2 μM, 5 μM, 10 μM or 15 μM, this treatment being continued throughout the step of multiplication of said melanocytes.

Said medium suitable for culturing melanocytes may in particular contain human FGF, hydrocortisone, bovine insulin and antibiotics or any other growth or differentiation factor compatible with melanocyte survival. Preferably, the M2 medium from Promocell (reference C24300) will be used.

Finally, freezing step c) may entail, for example:

centrifuging the melanocytes multiplied beforehand in the presence or absence of PTU and resuspending the pellet, for example in foetal calf serum +10% DMSO so as to obtain a concentration of 10⁶ cells/ml;

freezing said melanocytes in cryotubes at −1° C. per min down to −80° C.;

after 24 h, placing these tubes at −196° C. in liquid nitrogen.

These melanocytes may be thus conserved in a frozen form (for example: frozen library) for the purpose of subsequent use.

In the case of melanocytes derived from individuals of phototype IV, V or VI or from hyperpigmentary lesions, freezing step c) will advantageously be carried out in the presence of at least one compound selected from among 1-phenyl-3-(2-thiazolyl)-2-thiourea (PTU), a PTU analogue, a PTU mimetic and mixtures thereof.

During the subsequent use, the frozen tubes may be placed in a water bath at 37° C. for a very short period of time (thawing) and their content re-cultured in a medium suitable for culturing human melanocytes, as defined above.

The present invention also features a kit for producing a human melanocyte library, in particular a library of human melanocytes derived from individuals of phototype IV, V or VI or from hyperpigmentary lesions, comprising (i) a preparation of human melanocytes, in particular derived from individuals of phototype IV, V or VI or from hyperpigmentary lesions, and (ii) a culture medium for melanocytes supplemented with at least one compound selected from among PTU, a PTU analogue, a PTU mimetic and mixtures thereof.

In particular, the culture medium for melanocytes will contain at least growth factors (for example: FGF), antibiotics or antifungal agents (amphotericin B, gentamycin), insulin and hydrocortisone, may or may not contain serum, and may be supplemented according to the invention with PTU at a concentration that may range from 2 to 40 μM, preferably from 2 to 20 μM, and in particular a PTU concentration equal to 5 μM, 10 μM or 15 μM.

Also featured according to the invention is a frozen library of human melanocytes derived from phototype IV, V or VI or from hyperpigmentary lesions, obtained according to the method described above.

In particular, said library is characterized in that said melanocytes of phototype IV, V or VI exhibit, after thawing and re-culturing, a percentage viability that is increased by a factor of 10 to 100, in particular by a factor of from 10 to 20, compared with the percentage viability of non-treated melanocytes.

The percentage viability of highly pigmented melanocytes (for example: African) treated with an effective and non-cytotoxic amount of PTU of 5 μM or 10 μM may be, for example, at least 50%, and preferentially at least 70%. In particular, the percentage viability of the pigmented melanocytes (for example: African) treated with 10 μm PTU will be 80%, compared with the percentage viability of non-treated melanocytes, which is only 5% after thawing and re-culturing.

This percentage viability is determined through the percentage of cells that adhere to the culture support, or through any other known technique, for instance trypan blue staining, in which the non-adherent cells are stained.

These cultures of human melanocytes or these libraries of human melanocytes, in particular derived from individuals of phototype IV, V or VI or from hyperpigmentary lesions, can be used in the context of studies intended to supplement the knowledge regarding the biology and physiology of highly pigmented melanocytes under normal conditions, conditions of stress (UV, etc.) or pathological conditions (for example: hyperpigmentary disorders of the type lentigines, melasma, naevi, etc.).

Such cultures of human melanocytes or libraries of human melanocytes, in particular derived from individuals of phototype IV, V or VI, can also be used in tests for evaluating and/or for screening cosmetic active agents (for example: depigmenting agents) or pharmaceutical active agents.

These cultures of human melanocytes or libraries of human melanocytes may also be advantageously used for preparing co-cultures comprising at least melanocytes and keratinocytes, or for preparing reconstructed epidermides and/or reconstructed skin, that are in particular highly pigmented, and are themselves intended for the screening of active agents or else for the treatment of skin lesions, in particular in individuals with high phototypes.

This invention also features the use (i) of at least one compound selected from among 1-phenyl-3-(2-thiazolyl)-2-thiourea (PTU), a PTU analogue, a PTU mimetic and mixtures thereof, or (ii) of human melanocytes multiplied beforehand in the presence of PTU, in a method for obtaining co-cultures (for example: keratinocyte-melanocyte co-culture) or for obtaining reconstructed epidermides and/or reconstructed skin, in particular of phototype IV, V or VI.

Preferably, the human melanocytes multiplied beforehand in the presence of PTU will be used directly in a method for obtaining melanocyte and keratinocyte co-cultures, or for obtaining reconstructed epidermides and/or reconstructed skin, in particular of phototype IV, V or VI.

In particular, the invention relates to the use of highly pigmented human melanocytes derived from individuals of phototype IV, V or VI, that have been multiplied in the presence of PTU, in a method for obtaining melanocyte and keratinocyte co-cultures, or highly pigmented reconstructed epidermides and/or highly pigmented reconstructed skin.

The melanocyte and keratinocyte co-cultures are obtained according to the conventional methods. In particular, the co-culture will contain keratinocytes and melanocytes in a proportion of 10 to 1, more particularly of 3 to 1.

These keratinocyte-melanocyte co-cultures are useful for furthering the knowledge regarding melanocytes, in particular highly pigmented melanocytes, and especially as regards the formation of the epidermal melanization unit (keratinocyte-melanocyte contact) and the transfer of melanosomes to keratinocytes, under normal conditions, conditions of stress (UV, etc.) or pathological conditions (for example: hyperpigmentary disorders of the type lentigines, melasma, naevi, etc.).

In particular, the obtaining, according to the invention, of cultures, libraries or melanocyte-keratinocyte co-cultures from melanocytes derived from hyperpigmentary lesions may be useful for understanding the mechanisms associated with this hyperpigmentary disorder, and for the development of suitable treatments.

A method for preparing reconstructed epidermides and/or reconstructed skin, in particular reconstructed epidermides and/or reconstructed skin of phototype IV, V or VI, may comprise:

a) a step of preparation of a dermal support or of a dermal equivalent; and

b) a step of seeding onto said support at least (i) a preparation of keratinocytes and (ii) a preparation of human melanocytes, in particular derived from individuals of phototype IV, V or VI, said melanocytes having been multiplied beforehand in the presence of at least one compound selected from PTU, a PTU analogue, a PTU mimetic and mixtures thereof.

The reconstructed epidermides and/or reconstructed skin may also integrate other cell types, such as dermal cells (for example: fibroblasts), epidermal cells (for example: Langerhans cells, Merkel cells), or else hair cells (for example: dermal papilla), endothelial cells, nerve cells, sebocytes and/or adipocytes.

Step a) of preparation of a dermal support or of a dermal equivalent may be carried out according to the protocols described in (EP-A-285471, EP-A-285474, EP-A-789074, EP-A-502172, EP-A-418035, WO-A-9116010, EP-A-197090, EP-A-20753, FR-A-2665175, FR-A-2689904).

In particular, said dermal support or equivalent will be selected from among collagen/fibroblast lattices, a de-epidermalized dermis (DED) prepared as described in Régnier et al. (Front Matrix Biol., 9, 4-35,1981) and Régnier et al. (J. Invest. Dermatol., 109, 510-512,1997), a plastic bottle possibly coated with dermal macromolecules (collagen, fibronectin, laminin etc.) or artificial membranes of the biodressing for reconstructed epidermis type (BPER).

Preferably, the BPER-type EPISKIN® support or any other synthetic membrane, which is uncoated or coated with a film of dermal macromolecules, will be used, this support being able to possibly integrate dermal cells, for example fibroblasts, hair cells, for example dermal papilla, endothelial cells, nerve cells, sebocytes and/or adipocytes. A collagen sponges support on which are seeded keratinocytes, fibroblasts and endothelial cells is described for example in Black et al., FASEB, vol.12, pp:1331-1340, October 1998.

Very generally, reconstructed epidermis and/or reconstructed skin models consist of human keratinocytes deposited onto a support, often a dermal equivalent, and cultured under conditions such that they enter into a differentiation programme that result in the formation of an epidermal equivalent. Other cell types, such as Langherans cells can also be integrated (EP0789074), so as to reconstitute an epidermis and/or a skin very similar to natural tissues.

Step b) of seeding and culturing the reconstructed epidermis will be carried out in a culture medium suitable for culturing keratinocytes and melanocytes, that may in particular contain at least one mitogenic growth factor for keratinocytes [for example: epidermal growth factor (EGF) and/or keratinocyte growth factor (KGF)], insulin, hydrocortisone and an antibiotic (for example: gentamycin, amphotericin B). Advantageously, said medium may also comprise a pituitary extract, for example of bovine origin, epinephrine, transferrin, hydrocortisone or any other growth factor and/or nonessential amino acids.

Said medium may or may not contain serum and may eventually be supplemented with TGF-β growth factor.

The preparation of keratinocytes (i) may be obtained, for example, from a skin explant taken from an individual, according to the following protocol:

the subcutaneous tissue is removed using a scalpel;

the skin sample is decontaminated by means of an antibiotic treatment (for example: gentamycin);

the dermis is separated from the epidermis by a proteolytic treatment (for example: trypsin and dispase) and then dissection;

the dissociation of the cells is subsequently promoted in the presence of a solution of 0.05% trypsin and 0.02% EDTA; and the effect of the trypsin is neutralized by adding a DMEM culture medium containing 10% of serum;

the cell suspension is homogenized and is subsequently washed in culture medium for keratinocytes (KGM, Bullet kit, Clonetics Corp), or medium with serum and growth factors. The keratinocytes may or may not be cultured in the presence of feeder layers.

In addition, the preparation of melanocytes, in particular derived from individuals of phototype IV, V or VI (ii) may be obtained as described above according to the invention, in the presence or absence of an effective and non-cytotoxic amount of PTU.

According to an alternative embodiment, the PTU may also be added during the keratinocyte and melanocyte seeding step b), and maintained for a given period of time depending on the desired degree of pigmentation.

Specifically, it may be advantageous to maintain the PTU treatment during the reconstruction of the epidermal equivalent so as to obtain epidermal and/or skin equivalents that are less pigmented than the epidermal and/or skin equivalents of natural phototypes IV, V or VI. These less pigmented equivalents will in particular be more suitable for the screening of pro-pigmenting active agents, that are difficult to detect on epidermal equivalents of the natural phototypes IV, V or VI, and also for studies of UV-induced pigmentation and evaluation of the protective effect of sunscreens on epidermal equivalents of such phototypes.

The present invention also features a kit for producing an epidermis and/or a reconstructed skin, in particular an epidermis and/or a reconstructed skin particularly of phototype IV, V or VI, comprising (i) a dermal support, (ii) a preparation of keratinocytes and (iii) a preparation of human melanocytes, in particular derived from individuals of phototype IV, V or VI, preamplified in the presence of an effective and non-cytotoxic amount of PTU ranging from 2 to 20 μM, and in particular equal to 5 μM, 10 μM and 15 μM.

The dermal support (i) may be selected from a de-epidermalized dermis (DED) or a biodressing for a reconstructed epidermis (BPER) EPISKIN®, as described above, or any support as collagen sponges that may incorporate cells as dendritic cells, endothelial cells, nerve cells, fibroblasts, etc., as described for example in Black et al., FASEB, vol. 12, pp:1331-1340, October 1998.

A preparation of keratinocytes (ii) may be obtained as described above.

The preparation of melanocytes derived from individuals of phototype IV, V or VI (iii) may be obtained as described above, in the presence of an effective and non-cytotoxic amount of PTU.

According to an alternative embodiment, the kit may also contain (iv) an amount of PTU to be added at the time said keratinocytes and melanocytes are seeded onto the support, at a concentration ranging from 2 to 40 μM, so as to obtain reconstructed epidermides that are less pigmented.

The reconstructed epidermides and/or reconstructed skin, that are in particular highly pigmented, obtained according to the invention, may in particular be used in methods for evaluating and/or screening cosmetic and pharmaceutical active agents, in particular depigmenting or pro-pigmenting active agents, according to conventional methods. They may also be used for any study relating to UV-induced pigmentation, and to evaluation of the protective effectiveness of sunscreens, and of the effect of anti-pigmenting molecules.

Since the effect of the PTU is reversible, the melanocytes or reconstructed epidermides and/or reconstructed skin are also suited for the treatment of skin lesions of accidental origin (for example: third-degree burns), surgical origin (for example: naevus, tattoo) or pathological origin (for example: depigmentary disorders of the vitiligo type, or hyperpigmentary disorders of the type lentigines, melasma, naevi, etc.).

The individuals treated may be of any phototype; in particular, they will be of phototype IV, V or VI.

The treatment may entail:

preparing melanocytes from a skin explant from a normal individual (homologous cells) or from normal areas of the individual exhibiting lesioned skin (autologous cells);

multiplying said melanocytes in the presence of PTU, as described above;

re-implanting said melanocytes in the areas of lesioned skin, either in the form of melanocytes, or in the form of reconstructed epidermides and/or reconstructed skin containing said melanocytes.

In the specific case of a lesion of depigmentary type (for example: vitiligo) or hyperpigmentary type (for example: lentigines, melasma, naevi, etc.), the re-implantation of the melanocytes in the areas of depigmented skin may be carried out after abrasion of the epidermis in said depigmented areas.

This invention also features a method for screening and/or evaluating active agents capable of modulating the pigmentation of the skin, comprising at least one step in which said active agent is applied to human melanocyte cultures, or to reconstructed epidermides and/or reconstructed skin as obtained according to the invention.

In particular, this method features the screening and/or evaluating the depigmenting or pro-pigmenting activity of compounds.

According to a particular embodiment, the method for screening and/or evaluating the depigmenting activity of a product on melanocytes or reconstructed epidermides as obtained according to the invention may comprise the following steps: the test product is applied to said cell model or it is introduced into the culture medium, the luminance L* or lightness of said model is measured, and this measurement is compared with a control (without test product). For a depigmenting active agent, a greater luminance compared with the control will be obtained.

In order to further illustrate the present invention and the advantages thereof, the following specific examples are given, it being understood that same are intended only as illustrative and in nowise limitative. In said examples to follow, all parts and percentages are given by weight, unless otherwise indicated.

### BRIEF DESCRIPTION OF THE FIGURES OF DRAWING

FIG. 1 is a graphic representation of the measurement of the luminance, with a colorimeter, on African melanocytes in culture before freezing.

FIG. 2 is a graphic representation of the effect of an effective, non-toxic concentration of PTU of 10 μM on the in vitro multiplication of African melanocytes.

FIG. 3 is a graphic representation of the depigmenting activity of arbutin and of vitamin C phosphate evaluated on an African reconstructed epidermis prepared from African melanocytes cultured in the presence of PTU.

### EXAMPLES

### Example 1

### Effect of PTU on the Culture of Melanocytes of Phototype VI (African)

a) Preparation of a Primary Culture of African Melanocytes

The melanocytes were obtained from a foreskin of an African child according to the following protocol: the skin is cut into fragments of 6 mm and incubated at +4° C. overnight in the presence of 0.25% trypsin. The epidermis is then separated from the dermis in foetal calf serum. The pieces of dermis are scraped in order to recover the basal cells and the melanocytes. The epidermal fragments are then vortexed and the cells are then centrifuged at 190 G for 5 min. These cells (mixture of melanocytes and keratinocytes) are counted and seeded at a density of 8 to 15×10⁶ cells per 75 cm² flask, in 18 to 20 ml of M2 medium (Promocell).

The melanocytes are then purified according to the following protocol: the culture medium is changed 3 times a week and when the cells are confluent, the flasks are emptied and rinsed once with PBS —Ca²⁺ Mg²⁺. 3 ml of trypsin-EDTA are then added per flask and the flasks are incubated at ambient temperature for 3-4 min: the melanocytes detach whereas the keratinocytes remain attached to the flask. An equivalent volume of M2 medium is added and the detached cells are counted and seeded at a density of 10⁶ cells per 75 cm² flask, in 18 to 20 ml of M2 medium (Promocell). A primary culture of African human melanocytes is thus obtained.

The trypsinization is repeated when the melanocytes reach confluency (passage 1 et seq.).

The melanocytes in the primary culture are 80% viable, i.e., 80% of the melanocytes adhere to the flask.

b) Determination of the Non-Cytotoxic Concentration of PTU: Reversible Effect:

Part of the primary culture of melanocytes is treated with PTU (Sigma) at the concentrations of 5 μM, 10 μM and 50 μM, at passage 2/3 or preferably from the first passage.

The cytotoxicity of the effect of the PTU is evaluated by observing the cells under a microscope.

The results show that PTU is cytotoxic at a concentration of 50 μM on melanocytes from African individuals: most of the cells detach.

On the other hand, at concentrations of 5 μM and 10 μM, the cells remain 95% adherent (95% viability), and therefore a concentration 5 μM or 10 μM of PTU not only does not affect the viability of the melanocytes (decreases or not at all their viability), but is even capable of increasing their viability (95% versus 80% for the primary culture).

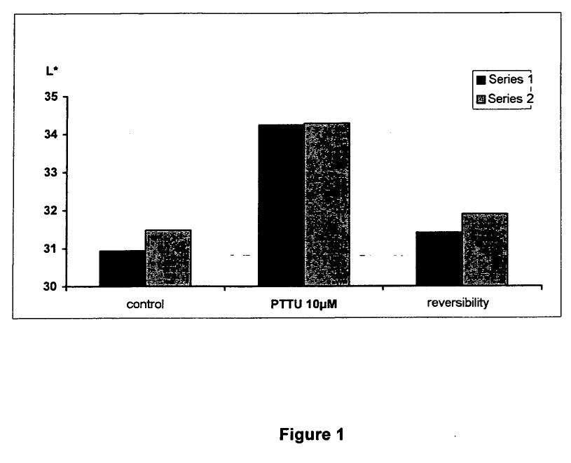

The pigmentation of the melanocytes of phototype VI (African), treated with 10 μM PTU or not treated, is measured by the value L* (luminance) of the cultures given by the spectrocolorimeter (FIG. 1). The results show that treatment of the cells with PTU at a concentration of 10 μM decreases the amount of pigment in the melanocytes and that this effect is reversible when the treatment is stopped. Similar results were obtained with a concentration of 5 μM.

The amount of pigment excreted by the melanocytes into the medium is also reduced after treatment. Melanin synthesis is decreased during treatment and becomes normal again in the absence of treatment.

The IC50 of PTU (i.e., the concentration of PTU for which 50% inhibition of melanogenesis is observed compared with the control on cultures of melanocytes of phototype VI (African), is evaluated from 50 μM to 100 μM.

The non-cytotoxic concentration of PTU defined according to the invention must therefore be less than the IC50 and must allow transient and reversible inhibition of melanin production. In particular, this non-cytotoxic concentration will be less than the IC50 by a factor of 1.5 to 10, in other words, at a concentration of from IC50/1.5 to IC50/10.

c) Effect of a Non-Cytotoxic Concentration of PTU on the Proliferation of Melanocytes of Phototype VI (African):

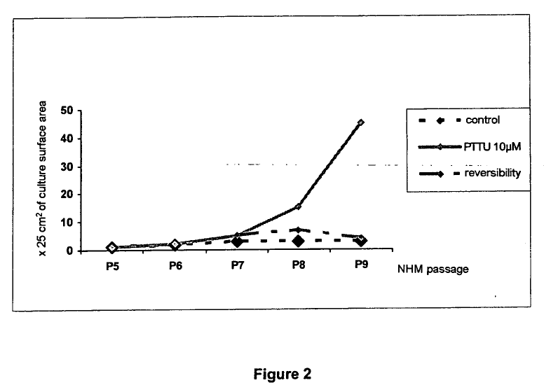

The effect of 10 μM PTU on melanocyte proliferation is estimated by the amount of cells obtained, as represented in FIG. 2.

The results show that the melanocytes of phototype VI (African) in culture, in the absence of PTU (control curve), are multiplied in a constant manner by a factor of 2 at each passage. In addition, their percentage viability is estimated at 80%.

The multiplication of the melanocytes of phototype VI (African) treated with 10 μM PTU at passage 4/5 is increased compared with the multiplication of the control melanocytes, from passage 6/7.

At passages 8 and 9, the treated cells are multiplied by a factor of 5 and by a factor of 15, respectively, compared with the non-treated control factor of 2. Their percentage viability is estimated at 95%.

The multiplication of the melanocytes of phototype VI (African) in the presence of PTU is therefore increased by a factor of 2 to 10 compared with the multiplication of the human melanocytes in the absence of PTU (control). This effect is reversible: after the treatment has been stopped at passage 7 (reversibility curve, FIG. 2), the proliferation is inhibited and similar to that of the non-treated cultures (10 μM PTU curve).

The same experiments reproduced with a concentration of 5 μM instead of 10 μM gave results of the same order of magnitude when the melanocytes are treated from the first passage.

d) Effect of a Non-Cytotoxic Concentration of PTU on the Viability of Melanocytes of Phototype VI (African) After Freezing:

The non-treated African melanocytes (80% viability) and the melanocytes treated with 10 μM PTU (95% viability) are frozen according to the following protocol: the cells are trypsinized and counted. They are then centrifuged and the pellet is resuspended in foetal calf serum +10% DMSO at a concentration of 10⁶ cells/ml.

The cells are frozen in cryotubes at −80° C.

After 24 h, the tubes are transferred into liquid nitrogen at −196° C.

For the thawing, the tubes are placed in a water bath at 37° C. and, after rapid thawing, 5 ml of M2 medium are added. The cells are centrifuged at 190 g for 5 min, the pellet is resuspended in M2 medium, and the cells are seeded into a flask and re-cultured under the conditions described in a).

At the end of these freezing, thawing and re-culturing steps, only 5% of the non-treated African melanocytes are viable, whereas 80% of the human melanocytes treated with 10 μM PTU are viable.

Similar results were obtained with a 5 μM PTU treatment.

The percentage viability of the melanocytes of phototype VI (African) after thawing and re-culturing is therefore increased by a factor of 10 to 20 for the African melanocytes treated with 5 μM or 10 μM PTU, compared with the percentage viability of the non-treated human melanocytes.

Other experiments showed that this factor could even be greater (10 to 100).

All these results therefore show that a non-cytotoxic concentration of PTU (for example: 5 or 10 μM) makes it possible (1) to promote and/or stimulate the multiplication of melanocytes of phototype VI (African) and (2) to maintain and/or increase their viability after freezing, thawing and re-culturing (for example: 80%, i.e., a percentage viability much greater than that obtained for non-treated African melanocytes, estimated to be 5%).

### Example 2

### Preparation of a Highly Pigmented Reconstructed Epidermis

The melanocytes multiplied in the presence of PTU can also be used in a method for obtaining a reconstructed epidermis as described below.

Unless otherwise indicated, all the media and buffers used are described in Régnier et al., Cell. Mol. Biol.; 45, 969-980, 1999; Duval et al., Pigment cell Res., 15, 440-446, 2002; Duval et al., Pigment cell Res., 14, 348-355, 2001.

The dermal supports or equivalents are prepared as described in Régnier et al., Front Matrix Biol., 9, 4-35, 1981; Régnier et al., J. Invest. Dermatol., 109, 510-512, 1997; Tinois et al., Exp. Cell Res., 193, 310-319, 1991; preferably, the biodressing for reconstructed epidermis (BPER) EPISKIN® support will be used, or any other synthetic membrane, uncoated or coated with a film of dermal macromolecules, it being possible for these membranes to be collagen sponges, dermal equivalents containing viable cells, fibroblasts, endothelial cells, nerve cells, adipocytes and/or sebocytes. A collagen biopolymer support on which are seeded keratinocytes, fibroblasts and endothelial cells is for example described in Black et al., FASEB, vol 12, pp:1331-1340, October 1998.

The support is rinsed in the culture medium and then possibly coated with collagen type IV, fibronectin or laminin, for example. After contact at +4° C. overnight, the keratinocytes and the melanocytes of phototype VI (African) multiplied beforehand in the presence of 5 μM or 10 μM PTU, as described in Example 1, are then seeded onto said support.

The culture can then be maintained immersed in a medium for keratinocytes in the presence of growth factor.

After an incubation time of 3 to 5 days, the skin equivalent of phototype VI (African) undergoing formation is maintained at the air/liquid interface, for example by deposition onto a metal screen or removal of the culture medium present at the surface of an insert. The liquid then preferentially consists of the same nutritive medium as the previous one, but in which the growth factors have been reduced.

The incubation then continues until a skin equivalent of phototype VI (African) is obtained.

Thus, the incubation continues for a period of from 5 to 30 days, preferentially from 7 to 10 days.

The reconstructed skin model of phototype VI (African) thus produced comprises two entities, the support, dermal equivalent, and the epidermal equivalent, which can be physically separated from one another.

The activity of the melanocytes of phototype VI (African) after the PTU treatment has been stopped is demonstrated by the DOPA reaction, which consists in revealing the action of the tyrosinase.

This enzyme is responsible for the oxidation of tyrosine to dihydrophenylalanine (DOPA) and then for the oxidation of DOPA to DOPA-quinone. The DOPA-oxidase activity of tyrosinase is demonstrated by the formation of a black precipitate, which is more intense as the melanocytes are more active.

The appearance of the African melanocytes, their localization and the transfer of the pigment are observed on histological sections after Fontana-Masson staining.

It is observed that the treated melanocytes, like the non-treated melanocytes, integrate into the reconstructed epidermis. They are in particular localized in the basal layer of the epidermis, and transfer melanin to the neighboring keratinocytes. In particular they are reactive with respect to the DOPA assay.

According to an alternative embodiment, the PTU treatment can also be maintained in the culture medium of the reconstructed epidermis in order to obtain reconstructed epidermides that are less pigmented, and more suitable for screening pro-pigmenting active agents and for evaluating UV-induced pigmentation and the protective effect of sunscreens and the anti-pigmenting effect of certain molecules.

### Example 3

### Use of the Pigmented Reconstructed Epidermides Obtained with PTU, as Models for Screening Depigmenting Active Agents

The epidermis reconstructed from melanocytes of phototype VI (African), prepared under the conditions of Example 2, is used to screen the depigmenting activity of active agents.

The test products—arbutin at 100 μM and vitamin C phosphate at 280 μM—are applied topically to said reconstructed epidermis: 3 applications (3 μM/sample) for 5 days from the 5th day of immersion.

Vitamin C phosphate at 280 μM is also applied systemically, i.e., in the culture medium, throughout the duration of the epidermal reconstruction.

The control is the measurement of luminance of the reconstructed epidermis in the absence of test product.

On epidermis reconstructed on BPER (Episkin®), since the dermal support is translucent, it cannot be evaluated directly on the samples. The latter are therefore de-epidermalized and then digested with a solution of proteinase K. The lysates obtained are filtered on DEAE cellulose, which retains the melanin. The luminance is then measured on the filter.

The luminance L*, or lightness, of the sample is measured using a colorimeter. The darker a sample is, the weaker its luminance.

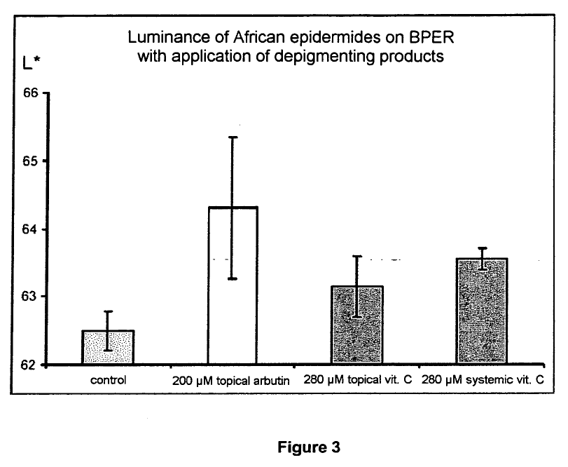

In the presence of arbutin or of vitamin C phosphate, an increase in luminance is observed (FIG. 3). The best result is observed with arbutin (increase of 1.8 points of L*).

These results therefore confirm the functionality of the pigmented reconstructed epidermides obtained from melanocytes and in the presence of PTU, as models for screening depigmenting active agents.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A method for promoting the in vitro multiplication of human melanocytes and/or the freezing thereof, comprising introducing into a culture of same, a thus effective amount of at least one 1-phenyl-3-(2-thiazolyl)-2-thiourea (PTU) compound or PTU analogue or PTU mimetic selected from the group consisting of vitamin C, arbutin, hydroquinone, kojic acid or acid or ester derivative thereof, ellagic acid, aminophenol derivative, procysteine or derivative thereof, niacinamide, isothiocyanate, thiocyanate, lucinol, and mixtures thereof.

2. The method as defined by claim 1, said human melanocytes being obtained from individuals of phototype IV, V or VI or from hyperpigmentary lesions.

3. The method as defined by claim 1, said melanocytes, for a given phototype, being obtained from one or more individuals of the same ethnic origin or of different ethnic origins.

4. The method as defined by claim 3, the sample of said melanocytes being obtained from the same anatomical site.

5. The method as defined by claim 1, wherein the amount of PTU, PTU analogue, PTU mimetic selected from the group consisting of vitamin C, arbutin, hydroquinone, kojic acid or acid or ester derivative thereof, ellagic acid, aminophenol derivative, procysteine or derivative thereof, niacinamide, isothiocyanate, thiocyanate, lucinol and mixtures thereof, present in the in vitro culture is provided according to following steps:
a) PTU, PTU analogue or PTU mimetic is applied to melanocytes in culture, in various amounts;
b) the cytotoxicity of the various amounts of PTU on the melanocytes in culture is evaluated by determining the percentage of cells that adhere to the culture support compared with untreated melanocytes and the amounts for which the percentage of cells that adhere to the culture support is greater than or equal to 60% are selected;
c) the effectiveness of the non-cytotoxic amounts of PTU selected in b), on the proliferation of melanocytes in culture, is evaluated by quantifying the number of cells obtained after each passage compared with untreated melanocytes, and the amounts of PTU for which an increase in the multiplication of said melanocytes by a factor of 2 to 10 is obtained, compared with untreated melanocytes, are selected.

6. The method as defined by claim 5, wherein the amount of PTU, PTU analogue, PTU mimetic selected from the group consisting of vitamin C, arbutin, hydroquinone, kojic acid or acid or ester derivative thereof, ellagic acid, aminophenol derivative, procysteine or derivative thereof, niacinamide, isothiocyanate, thiocyanate, lucinol and mixtures thereof, contacted with the human melanocytes before freezing, increases the percentage viability of said melanocytes, after thawing and re-culturing, by a factor of 10 to 20, compared with the percentage viability of untreated human melanocytes.

7. The method as defined by claim 5, wherein the amount of PTU, PTU analogue, PTU mimetic selected from the group consisting of vitamin C, arbutin, hydroquinone, kojic acid or acid or ester derivative thereof, ellagic acid, aminophenol derivative, procysteine or derivative thereof, niacinamide, isothiocyanate, thiocyanate, lucinol and mixtures thereof, ranges from IC50/1.5 to IC50/10.

8. The method as defined by claim 1, wherein the PTU is present in the in vitro culture in an effective amount ranging from 2 μM to 40 μM.

9. In a method for obtaining human melanocyte cultures or human melanocyte libraries, the improvement which comprises incorporating therein a thus effective amount of at least one 1-phenyl-3-(2-thiazolyl)-2-thiourea (PTU) compound or PTU analogue or PTU mimetic selected from the group consisting of vitamin C, arbutin, hydroquinone, kojic acid or acid or ester derivative thereof, ellagic acid, aminophenol derivative, procysteine or derivative thereof, niacinamide, isothiocyanate, thiocyanate, lucinol, and mixtures thereof.

10. The method as defined by claim 9, comprising obtaining primary or secondary melanocyte cultures or libraries.

11. In a method for obtaining co-cultures comprising at least melanocytes and keratinocytes, the improvement which comprises incorporating therein human melanocytes multiplied in the presence of a thus effective amount of at least one 1-phenyl-3-(2-thiazolyl)-2-thiourea (PTU) compound or PTU analogue or PTU mimetic selected from the group consisting of vitamin C, arbutin, hydroquinone, kojic acid or acid or ester derivative thereof, ellagic acid, aminophenol derivative, procysteine or derivative thereof, niacinamide, isothiocyanate, thiocyanate, lucinol, and mixtures thereof.

12. A reconstructed epidermide and/or reconstructed skin, comprising an amount of human melanocytes multiplied in the presence of a thus effective amount of at least one 1-phenyl-3-(2-thiazolyl)-2-thiourea (PTU) compound or PTU analogue or PTU mimetic selected from the group consisting of vitamin C, arbutin, hydroquinone, kojic acid or acid or ester derivative thereof, ellagic acid, aminophenol derivative, procysteine or derivative thereof, niacinamide, isothiocyanate, thiocyanate, lucinol, and mixtures thereof.

13. A regime or regimen for the treatment of skin lesions, comprising topically applying onto the affected skin area of an individual in need of such treatment, melanocytes or reconstructed epidermide and/or reconstructed skin comprised thereof multiplied in the presence of a thus effective amount of at least one 1-phenyl-3-(2-thiazolyl)-2-thiourea (PTU) compound or PTU analogue or PTU mimetic selected from the group consisting of vitamin C, arbutin, hydroquinone, kojic acid or acid or ester derivative thereof, ellagic acid, aminophenol derivative, procysteine or derivative thereof, niacinamide, isothiocyanate, thiocyanate, lucinol, and mixtures thereof.

14. The regime or regimen as defined by claim 13, said skin lesions comprising burns, skin excisions, depigmentations or hyperpigmentations of the skin.

15. The regime or regimen as defined by claim 13, said melanocytes having been obtained from normal individuals or from normal areas of individuals afflicted with said skin lesions.

16. The method as defined by claim 1, comprising introducing into said culture a thus effective amount of a PTU analogue which comprises a thiourea, a phenyl monoxide or dioxide, or a compound of formula (I): 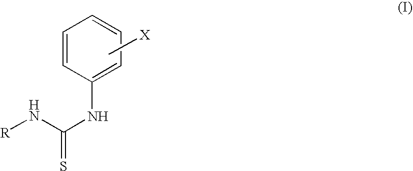
wherein:
X is a hydrogen atom or a linear or branched C₁-C₄ alkyl radical or a halogen or a cyano group or a radical —OR′ or COOR′ in which R′ is a hydrogen atom or a C₁-C₄ alkyl radical, and
R is a hydrogen atom or an aromatic group that is unsubstituted or substituted with groups equivalent to X.

17. The method as defined by claim 1, comprising introducing into said culture a thus effective amount of 1-phenyl-3-(2-thiazolyl)-2-thiourea.

18. The method as defined by claim 1, comprising introducing into said culture a thus effective amount of a PTU mimetic.

19. A human melanocyte culture or a human melanocyte library, comprising melanocytes multiplied in the presence of a thus effective amount of at least one 1-phenyl-3-(2-thiazolyl)-2-thiourea (PTU) compound or PTU analogue or PTU mimetic selected from the group consisting of vitamin C, arbutin, hydroquinone, kojic acid or acid or ester derivative thereof, ellagic acid, aminophenol derivative, procysteine or derivative thereof, niacinamide, isothiocyanate, thiocyanate, lucinol, and mixtures thereof, and thereafter optionally freezing same.

20. The human melanocyte culture or human melanocyte library as defined by claim 19, said melanocytes having been twice multiplied and twice frozen.

21. A kit for producing a culture or a library of human melanocytes obtained from individuals of phototype IV, V or VI or from hyperpigmentary lesions, comprising (i) a preparation of human melanocytes obtained from individuals of phototype IV, V or VI or from hyperpigmentary lesions, and (ii) a culture medium for melanocytes supplemented with an amount of PTU ranging from 2 to 40 μM.

22. A method for preparing reconstructed epidermides and/or reconstructed skin of phototype IV, V or VI, comprising:
a. a step of preparation of a dermal support or of a dermal equivalent; and
b. a step of seeding onto said support at least (i) a preparation of keratinocytes and (ii) a preparation of human melanocytes obtained from individuals of phototype IV, V or VI, said melanocytes having been preamplified in the presence of PTU, a PTU analogue, a PTU mimetic selected from the group consisting of vitamin C, arbutin, hydroquinone, kojic acid or acid or ester derivative thereof, ellagic acid, aminophenol derivative, procysteine or derivative thereof, niacinamide, isothiocyanate, thiocyanate, lucinol and mixtures thereof.

23. The method as defined by claim 22, further comprising adding at least one 1-phenyl-3-(2-thiazolyl)-2-thiourea (PTU) compound or PTU analogue or PTU mimetic selected from the group consisting of vitamin C, arbutin, hydroquinone, kojic acid or acid or ester derivative thereof, ellagic acid, aminophenol derivative, procysteine or derivative thereof, niacinamide, isothiocyanate, thiocyanate, lucinol, and mixtures thereof, during said seeding step b).

24. A kit for producing an epidermis and/or a reconstructed skin of phototype IV, V or VI, comprising (i) a dermal support or equivalent, (ii) a preparation of keratinocytes and (iii) a preparation of human melanocytes derived from individuals of phototype IV, V or VI, said melanocytes having been preamplified in the presence of an amount of PTU ranging from 2 to 40 μM.

25. In a method for screening and/or evaluating active agents capable of modulating the pigmentation of the skin, the improvement which comprises at least one step in which said active agent is introduced to a human melanocyte culture as defined by claim 19.

26. In a method for screening and/or evaluating active agents capable of modulating the pigmentation of the skin, the improvement which comprises at least one step in which said active agent is introduced to reconstructed epidermides and/or reconstructed skin obtained according to the method of claim 22.

27. A co-culture containing at least melanocytes and keratinocytes, comprising human melanocytes multiplied in the presence of a thus effective amount of at least one 1-phenyl-3-(2-thiazolyl)-2-thiourea (PTU) compound or PTU analogue or PTU mimetic selected from the group consisting of vitamin C, arbutin, hydroquinone, kojic acid or acid or ester derivative thereof, ellagic acid, aminophenol derivative, procysteine or derivative thereof, niacinamide, isothiocyanate, thiocyanate, lucinol, and mixtures thereof.
